(19) United States
(12) Patent Application Publication
(10) Pub. No.: US 2026/0021026 A1
(43) Pub. Date: Jan. 22, 2026

(54) MINERAL FORMULATION

(71) Applicant: Formulated Solutions, LLC, Largo, FL (US)

(72) Inventors: Michael Favero, Tampa, FL (US); Claire Matawaran, Clearwater, FL (US); Renee Nelson, Brandon, FL (US)

(73) Assignee: Formulated Solutions, LLC, Largo, FL (US)

(21) Appl. No.: 19/264,360

(22) Filed: Jul. 9, 2025

Related U.S. Application Data
(60) Provisional application No. 63/668,854, filed on Jul. 9, 2024.
(60) Provisional application No. 63/751,922, filed on Jan. 31, 2025.

(51) Int. Cl. A61K 8/27; A61K 8/04; A61K 8/25; A61K 8/29; A61K 8/37; A61K 8/73; A61Q 17/04

(52) U.S. Cl. CPC A61K 8/27; A61K 8/046; A61K 8/25; A61K 8/29; A61K 8/37; A61K 8/731; A61K 8/732; A61Q 17/04

(57) ABSTRACT

A formulation to be dispensed from a container system without void space capable of atomization of a homogeneous suspension containing a mineral compound(s) in particulate form, porous hydrophilic colloidal silica in particulate form, and water droplets absorbed/adsorbed within porous hydrophilic colloidal silica contained within an oleophilic diluent system; wherein the water droplets are not emulsified in the suspension; wherein the water droplets are within the porous hydrophilic colloidal silica in particulate form; wherein the suspension comprises a tightly packed solid dispersion of particulate matters exhibiting repulsive effects, keeping the mineral compound in particulate form from settling or sagging under gravimetric forces.

### TECHNICAL FIELD

This disclosure relates to the field of formulations containing mineral-based active ingredients applied topically by means of spray aerosolization or atomization.

### BACKGROUND

Specifically in relation to sunscreens, mineral actives such as zinc oxide and titanium oxide are currently believed to be environmentally superior in comparison to chemical alternatives currently available under FDA OTC regulations. Consequently, their use in sunscreen and other formulations is desirable.

The mineral compound zinc oxide is also utilized as a topically applied skin protectant. As the product is applied to compromised skin, a hand-free application method is desired.

However, due to the inherent insolubility of mineral actives, formulations with mineral actives must take the form of a suspension. The general way of stabilizing suspensions is by thickening the diluents or designing the product to be ‘shake-well prior to use’. Unfortunately, aerosolized formulations cannot be thickened beyond approximately 2000 cps without greatly inhibiting the spray-ability of the product. Additionally, Bag on Valve products cannot be ‘shake-well prior to use’. This is due to Bag on Valve aerosolized products lacking any void space within the product bag, which is necessary to appropriately resuspend material through shaking.

### SUMMARY

A formulative method is provides to stabilize the active mineral compounds in a manner that allows consumers to apply them for spray aerosolization or atomization.

In one aspect, this disclosure provides a formulation comprising a homogeneous suspension of a uniform composition of solid particulates comprising zinc oxide or titanium dioxide, or a combination of both, suspended in an oleophilic system possessing a positive ‘Octanol Water Partition Coefficient’, wherein the formulation does not comprise an emulsifier, wherein the formulation is expelled from a dispensing container system without void space, wherein said suspension does not comprise particulate settling of the homogeneous suspension of a uniform composition, wherein 70% or more of the solid particulates remains suspended through at least six months undisturbed.

In another aspect, this disclosure provides a dispensing container system without void space comprising a formulation comprising a homogeneous suspension of a uniform composition of solid particulates that includes zinc oxide, or titanium dioxide, or a combination of both, suspended in an oleophilic system possessing a positive ‘Octanol Water Partition Coefficient’, wherein the formulation does not comprise an emulsifier, wherein the formulation is expelled from a dispensing container system without void space, wherein said suspension does not comprise particulate settling of the homogeneous suspension of a uniform composition wherein more than 70% of the solid particulates remains suspended through at least six months undisturbed.

In another aspect, this disclosure provides a method of preparing a formulation, comprising:

Adding oils and emollient esters (Capric Caprylic Triglyceride/C₁₂₋₁₅ Alkyl Benzoate/Coco-
Caprylate) to a vessel;
applying a vigorous vortex using an impeller mixer;
allowing no less than two minutes of mixing between each addition;
Adding Mineral dispersants to the vessel;
Heating the vessel to 50° C.;
Homogenizing the vessel using a medium sized stator for two minutes at 7000 rpm;
Stopping homogenizing and returning vessel to vigorous prop mixing;
Adding suspending solids and rheology modifiers to the vessel;
allowing no less than two minutes of mixing between each addition;
Returning vessel to homogenizing using a medium sized stator for two minutes at 7000 rpm;
Stopping homogenizing and returning vessel to vigorous prop mixing;
Adding active ingredients (Zinc Oxide/Titanium Dioxide);
Allowing no less than two minutes of mixing between each addition;
Returning vessel to homogenizing using a medium sized stator for five minutes at 7000 rpm;
Stopping homogenizing and returning vessel to vigorous prop mixing;
Adding hydrophilic ingredients including fumed silica and ionized (salted) water to the vessel;
Allowing no less than two minutes of mixing between each addition;
Returning to homogenizing using a medium sized stator for five minutes at 7000 rpm;
Stopping homogenizing;
Using an anchor mixer under low shear, mixing until batch cools from 50° C. to room
temperature; thereby completing bulk processing and resulting in bulk;
Filling bulk into BOV systems at 35-40 psi cut bag pressure.

Numerous other aspects are provided in accordance with these and other aspects of the invention. Other features and aspects of the present invention will become more fully apparent from the following detailed description and the appended claims.

### DETAILED DESCRIPTION

As used herein, the word “a” or “plurality” before a noun represents one or more of the particular nouns. For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should be considered to include the end points 5 and 10.

The feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, controls.

The term “dispensing container system” is the same as a package, a can, a dispenser, etc.

The term “octanol-water partition coefficient” is known in the art. Briefly, it refers to a measure of how a substance distributes itself between a phase of n-octanol (an organic solvent) and water, essentially indicating how “fat-soluble” or “water-soluble” a compound is.

Due to continually tightening restrictions regarding chemical sunscreen actives and a need for improving the environment, the desire for mineral sunscreen actives has increased, with respect at least to sunscreens. However, due to their inherent insolubility, formulations utilizing mineral sunscreen actives must take the form of a suspension. The general way of stabilizing suspensions is by thickening the diluents. Unfortunately, this method of stabilizing is difficult to achieve within the desired Bag on Valve (BOV) packaging system, which for many brand owners has been identified as a consumer-preferred manner of dispensing sunscreens due the absence of liquid aerosol propellant systems.

BOV is a dispensing container system. A type of aerosol, BOV systems pressurize the void space within a can containing a bag or pouch filled with product. When the aerosol button is depressed, the pressure exerted on the bag forces product into the actuator and is subsequently expelled as a targeted stream, an atomized mist or anywhere between. In a BOV system, the expelled product spray pattern is primarily controlled by the pressure surrounding the bag, the actuator configuration, and the physical properties of the product, mainly its viscoelastic properties. If the product viscosity is thick, little can be done to decrease spray pattern droplet size. This effectively removes thickening as a viable method of stabilization within a BOV system.

Zinc oxide is insoluble in both common aqueous and oleophilic systems. Therefore, its use requires a homogenous suspension. For zinc oxide, this is most easily achieved by means of ‘shake-well’ just prior to use to resuspend settled zinc oxide particulate. Unfortunately, this method cannot be utilized with a BOV system as the absence of void space within the pressurized bag greatly reduces the effects of shaking. Therefore, permanent suspension is required when using a BOV system or other comparable system that works to contain and pressurize a contained formula by means of direct physical compression (ex. Airopack, piston can, compression sleeve BOV, rubber tube over the bag, etc.).

In one aspect, this disclosure provides a formulation comprising a homogeneous suspension of a uniform composition of solid particulates comprising zinc oxide or titanium dioxide, or a combination of both, suspended in an oleophilic system possessing a positive ‘Octanol Water Partition Coefficient’, wherein the formulation does not comprise an emulsifier, wherein the formulation is expelled from a dispensing container system without void space, wherein said suspension does not comprise particulate settling of the homogeneous suspension of a uniform composition, wherein more than 70% of the solid particulates remains suspended through at least six months undisturbed. In further embodiments, more than 70% of the solid particulates remains suspended through one year undisturbed.

In another aspect, this disclosure provides a dispensing container system without void space comprising a formulation comprising a homogeneous suspension of a uniform composition of solid particulates that comprises zinc oxide, or titanium dioxide, or a combination of both, suspended in an oleophilic system possessing a positive ‘Octanol Water Partition Coefficient’, wherein the formulation does not comprise an emulsifier, wherein the formulation is expelled from a dispensing container system without void space, wherein said suspension does not comprise particulate settling of the homogeneous suspension of a uniform composition wherein more than 70% of the solid particulates remains suspended through at least six months undisturbed. In some embodiments, more than 70% of the solid particulates remains suspended through one year undisturbed.

In another aspect, this disclosure provides a method of preparing a formulation, comprising:

Adding oils and emollient esters (Capric Caprylic Triglyceride/C₁₂₋₁₅ Alkyl Benzoate/Coco-
Caprylate) to a vessel;
applying a vigorous vortex using an impeller mixer;
allowing no less than two minutes of mixing between each addition;
Adding Mineral dispersants to the vessel;
Heating the vessel to 50° C.;
Homogenizing the vessel using a medium sized stator for two minutes at 7000 rpm;
Stopping homogenizing and returning vessel to vigorous prop mixing;
Adding suspending solids and rheology modifiers to the vessel;
allowing no less than two minutes of mixing between each addition;
Returning vessel to homogenizing using a medium sized stator for two minutes at 7000 rpm;
Stopping homogenizing and returning vessel to vigorous prop mixing;
Adding active ingredients (Zinc Oxide/Titanium Dioxide);
Allowing no less than two minutes of mixing between each addition;
Returning vessel to homogenizing using a medium sized stator for five minutes at 7000 rpm;
Stopping homogenizing and returning vessel to vigorous prop mixing;
Adding hydrophilic ingredients including fumed silica and ionized (salted) water to the vessel;
Allowing no less than two minutes of mixing between each addition;
Returning to homogenizing using a medium sized stator for five minutes at 7000 rpm;
Stopping homogenizing;
Using an anchor mixer under low shear, mixing until batch cools from 50° C. to room
temperature; thereby completing bulk processing and resulting in bulk;
Filling bulk into BOV systems at 35-40 psi cut bag pressure.

This disclosure provides a method of preparing a formulation comprising mineral compounds, comprising:
(a) providing an Oil Continuous Phase comprising homogenizing oil(s), ester(s), polysiloxane(s), and/or triglyceride(s);
(b) adding inorganic sunscreen including mineral compounds in particulate form and continuing homogenization resulting in homogenate;
(c) transferring homogenate from (b) to low shear mixing and adding hydrophilic colloidal silica, ionizable liquid, and ion contributor and continuing homogenization.
   In further embodiments, the method further comprises (d) adding fragrance under low shear mixing at room temperature.

This disclosure provides a dispensing container system without void space comprising a formulation comprising a homogeneous suspension comprising suspended mineral compounds in particulate form, porous hydrophilic colloidal silica in particulate form and water droplets absorbed into porous hydrophilic colloidal silica contained within an oleophilic diluent system; wherein the water droplets are not emulsified in the suspension; wherein the water droplets are within the porous hydrophilic colloidal silica in particulate form; wherein the suspension comprises a tightly packed solid dispersion of particulate matters exhibiting repulsive effects, keeping the mineral compound in particulate form from settling or sagging under gravimetric forces.

A formulation is provided comprising a homogeneous suspension comprising suspended mineral compounds in particulate form, porous hydrophilic colloidal silica in particulate form; and water droplets absorbed into porous hydrophilic colloidal silica contained within an oleophilic diluent system; wherein the water droplets are within the porous hydrophilic colloidal silica in particulate form; wherein the suspension comprises a tightly packed solid dispersion of particulate matters exhibiting repulsive effects, keeping the mineral compound in particulate form from settling or sagging under gravimetric forces; wherein the formulation is expelled from a dispensing container system without void space. This disclosure also provides a formulation expelled by a disclosed container dispensing system. This disclosure provides a formulation produced by a disclosed method.

In certain embodiments, the formulation comprises one or more solid particulates selected from the group consisting of hectorite (including stearalkonium hectorite gel), bentonite, iron oxide, mica, hydroxyapatite, kaolin, clay, stearalkonium hectorite, and distarch phosphate. In some embodiments, the formulation comprises stearalkonium hectorite gel.

In some embodiments, coco-caprylate and/or C₁₂-C₁₅ Alkyl Benzoates are present within the outer phase of the formulation. In further embodiments, coco-caprylate, and/or C₁₂-C₁₅ Alkyl Benzoates are utilized above 20% formula weight due to their superior atomization capabilities. In some embodiments, the formulation comprises coco-caprylate.

In some embodiments, water containing chloride salts or sulfate salts are present within the formulation inner phase below 20% formula weight. In some embodiments, the formulation comprises 6.5% to 18% saltwater. In further embodiments, the formulation comprises 11% to 13% saltwater. In yet further embodiments, the formulation comprises 12.6% saltwater.

In some embodiments, the formulation comprises triethoxycaprylylsilane coated zinc oxide and/or titanium dioxide.

In some embodiments, the zinc oxide is present from 0% to 40% of the formulation's weight (0% w to 40% w) and the titanium dioxide is present from 0% to 25% of the formulation's weight. In some embodiments, the formulation comprises SPF 30+ results and comprises 20% w to 25% w zinc oxide. In some embodiments, the mineral compounds is at about 5% to about 40% of the formulation's weight.

In some embodiments, the dispensing container system does not need to be shaken before the formulation is expelled from the dispensing container system. In some embodiments, the formulation is for topical, cosmetic, drug, or medical device application.

In some embodiments, the dispensing container system without void space is a bag on valve (BOV) pressurized assembly package, comprising a two-way fill/dispensing valve, an attached internal high barrier bag affixed to said valve, and rigid container adapted to holding positive pressure affixed to the valve. In further embodiments, the dispensing container system is glass, barrier resin, metal/alloy, or another material capable of holding positive pressure. In some embodiments, the pressurized assembly expels the formulation in a metered dispensing system.

In some embodiments, the dispensing container system without void space is an Airopack system, a piston can system, a compression sleeve BOV system, or a rubber tube over bag system.

In some embodiments, the suspension does not comprise particulate settling of the mineral sunscreen compound above about 10% formulation weight. In some embodiments, the formulation's average aerosolized droplet size is less than 50 microns using a volume distribution measured by laser diffraction or microscopy. In some embodiments, the formulation expelled from the dispensing container system are droplets that are less than about 20 microns in diameter. In some embodiments, the dispensing container system does not need to be shaken before the formulation is expelled from the dispensing container system. In some embodiments, the formulation is for topical, cosmetic, drug, or medical device application.

In some embodiments, the formulation comprises the following ingredients in the following pe

| | | Lower | Upper |
|---|---|---|---|
| | Specific Ingredients | % Weight | % Weight |
| Ingredient Class | (N = 2 Provided per Class) | Limit | Limit |
| Emollient Ester | C₁₂₋₁₅ Alkyl Benzoate/ | 20.0 | 90.0 |
| | Coco-Caprylate | | |
| Mineral Dispersant | Polyhydroxystearic Acid/ | 0.5 | 10.0 |
| | Polyamide-8 | | |
| Oil Suspension Aid | Steralkonium Hectorite/ | 0.2 | 10.0 |
| | Ethyl Cellulose | | |
| Mineral Active | Zinc Oxide/ | 5.0 | 40.0 |
| | Titanium Dioxide | | |
| Hydrophilic | Fumed Silica/ | 0.2 | 5.0 |
| Suspension Aid | Dibasic Calcium Phosphate | | |
| Hydrophilic Diluent | Water/ | 2.0 | 20.0. |
| | Glycerin | | |

In some embodiments, the formulation comprises one or a subset of these ingredients in the these percentages.

In some embodiments, the formulation is for topical application. In further embodiments, the formulation is a sunscreen. In yet further embodiments, the sunscreen formulation further comprises one or more tinting agents.

In some embodiments, the triglyceride/oil comprises capric caprylic triglyceride. In some embodiments, the dispersing agent is inorganic and comprises polyhydroxystearaic acid. In some embodiments, the oleophilic thickener comprises dextrin palmitate and ethylhexanoate. In some embodiments, the shear thinning viscosity modifier comprises steralkonium hectorite. In some embodiments, the water-resistant former comprises capryloyl glycerin/sebacic acid copolymer. In some embodiments, the SPF booster comprises pongamia seed extract.

In some embodiments, the formulation is an inorganic sunscreen, and the sunscreen includes mineral compounds in particulate form comprising zinc oxide or titanium oxide, or both.

In some embodiments, the mineral compound skin protectant or inorganic sunscreen in particulate form is coated or chemically bonded in a hydrophobic material, an inorganic dispersing agent, or both. These coatings are comprised of triethoxycaprylylsilane, triethoxycaprylylsilane ethyl ferulate, stearic acid, jojoba esters, hydrogen methicone, stearoyl glutamic acid, and/or polysiloxanes.

In some embodiments, the hydrophilic colloidal silica comprises hydrophilic fumed silica; and/or the ionizable liquid comprises water; and/or the ion contributor comprises sodium chloride and/or magnesium sulfate.

In some embodiments, the method further comprises adding one or more color shifting agents including iron oxides, Mica's, and water insoluble dyes.

In certain embodiments, the water droplets dispersed within an oleophilic diluent system can be thought of as being dispersed in an oleophilic ‘outer phase’ diluent system.

In certain embodiments, to achieve atomization upon BOV actuation without high viscosity this disclosure utilizes repulsion stabilization. This form of stabilization utilizes the lack of void space within a BOV system to its advantage. In certain embodiments, the disclosed formulation has large volume of suspended particulate; thus each individual particulate would need to ‘push’ other solid particulate out of the way in order to settle. Because there are no void or gaseous spaces within, for example, a BOV system, there is nowhere for particulate to be ‘pushed,’ leaving condensing the space between particles the only option. However, as the particulate carry ionic repulsive effects the solid particulates do not like to be condensed. The resulting effect is a stable suspension of zinc oxide within a pressurized, for example, BOV system.

In some embodiments, this disclosure provides a bulk suspension containing zinc oxide particulate for use within a Bag on Valve (BOV) aerosol system to deliver zinc oxide and other dense, insoluble particulate in a spray which may be characterized as misting or atomizing.

The disclosed formulation, system and method can be made to utilize zero greenhouse or ozone depleting gases. The disclosed formulation, system and method can drastically lower flammability/combustibility concerns associated with traditional zinc oxide aerosol products. The disclosed formulation, system and method can be made to be a minimum of 90% ISO16128 natural origin. The disclosed formulation, system and method can work with formulations containing large concentrations of solid particulate (5%-40% formulation weight). The disclosed formulation, system possesses a viscosity less than 10,000 centipoise using a T-bar spindle@5 RPM with helipath turned on resulting in a consumer preferred small droplet, misting spray rather than large globules attributable to spraying highly viscous material. The hydrophobicity of the disclosed formulation can improve the resulting water resistance of applied material.

In some embodiments, repulsion stabilization is utilized to achieve a suspension absence of particulate settling of zinc oxide in quantities above 5% of the formulation's weight.

In some embodiments, the formulation is for topical, cosmetic, drug, or medical device application. In some embodiments, the disclosed formulation is a skin protectant and may be used to prevent or treat diaper rash. In other embodiments, the disclosed formulation is a sunscreen formulation.

In certain embodiments, the disclosed formulation does not require vigorous shaking prior to use when contained within a Bag on Valve (BOV) aerosol system. Currently, several water out emulsion systems containing zinc oxide and/or titanium dioxide exist within a BOV aerosol format. However, all carry a ‘shake well’ label. Ideally doing so helps to resuspend minerals which have settled out during an active stretch of time. Such shaking would do little to appropriately resuspend settled minerals within a BOV format. This is due to the unique construction of a BOV aerosol system which does not allow head space or void space with the BOV bag. As a result, shaking does little in comparison to a traditional container, including aerosols which contain a void space occupied by gaseous compounds and allowing space for liquids and solids to move and mix.

In some embodiments, the disclosed formulation is for use within an aerosolized BOV system to deliver a fine mist of zinc oxide for either barrier protection or sun protection. In some embodiments, the disclosed formulation is a stable, long-term suspension of zinc oxide that does not require agitation or energy to resuspend. In some embodiments, the disclosed formulation comprises Zinc Oxide % weight range of 10%- 40%. In further embodiments, the zinc oxide % weight is 20%. In some embodiments, the disclosed formulation The ability to be dispensed from an aerosolized container system, specifically a BOV and produce a subsequent fine mist. In some embodiments, the disclosed formulation possesses a broad-spectrum SPF value of 30+.

In some embodiments, the disclosed formulation contains one or more of a wide range of additional, optional components. For example, the CTFA Cosmetic Ingredient Handbook, Seventh Edition, 1997, the Eighth Edition, 2000, and the Personal Care Council website, describe a wide variety of cosmetic and pharmaceutical ingredients commonly used in skin care formulations, which are suitable for use in the disclosed formulations. Examples of these functional classes disclosed in these references include, for example and without limitation: absorbents, abrasives, anticaking agents, anti-foaming agents, antioxidants, binders, biological additives, buffering agents, bulking agents, chelating agents, chemical additives, colorants, cosmetic astringents, cosmetic biocides, cryoprotectants, film stabilizers, denaturants, drug astringents, external analgesics, film formers, fragrance components, humectants, pacifying agents, pH adjusters, plasticizers, preservatives, propellants, reducing agents, skin bleaching agents, skin-conditioning agents (emollients, humectants, miscellaneous, and occlusive), skin protectants, solvents, SPF enhancers/boosters, foam boosters, hydrotropes, solubilizing agents, suspending agents (nonsurfactant), sunscreen agents, ultraviolet light absorbers, water-proofing agents, and viscosity increasing agents (aqueous and nonaqueous).

### EXAMPLES

For this invention to be better understood, the following examples are set forth. These examples are for purposes of illustration only and are not to be construed as limiting the scope of the invention in any manner.

### Example 1. Exemplary Formulation

Table 1 Provides an exemplary formulation.

TABLE 1

| Ingredient | % Weight |
|---|---|
| Capric Caprylic Triglyceride | 20.0 |
| Coco-Caprylate | 40.0 |
| Polyhydroxystearic Acid | 2.0 |
| Dextrin Palmitate/Ethylhexanoate | 0.5 |
| Steralkonium Hectorite | 3.0 |
| Capryloyl Glycerin/Sebacic Acid Copolymer | 1.0 |
| Zinc Oxide + Triethoxycaprylylsilane Ethyl Ferulate | 20.0 |
| Hydrophilic Fumed Silica | 1.0 |
| Water | 12.0 |
| Sodium Chloride | 0.5 |
| TOTAL: | 100.0 |

### Example 2. Aspects of the Disclosed Formulation

For this invention to be better understood, the following 2 tables have been set forth. Table 2 and Table 3 are intended to be read and understood as a pair rather than individual collections of data or statements.

TABLE 2
ranges which would provide the desired
effect in conjunction with Table 3.

| | | Lower | Upper |
|---|---|---|---|
| | Specific Ingredients | % Weight | % Weight |
| Ingredient Class | (N = 2 Provided per Class) | Limit | Limit |
| Emollient Ester | C₁₂₋₁₅ Alkyl Benzoate/ | 20.0 | 90.0 |
| | Coco-Caprylate | | |
| Mineral Dispersant | Polyhydroxystearic Acid/ | 0.5 | 10.0 |
| | Polyamide-8 | | |
| Oil Suspension Aid | Steralkonium Hectorite/ | 0.2 | 10.0 |
| | Ethyl Cellulose | | |
| Mineral Active | Zinc Oxide/ | 5.0 | 40.0 |
| | Titanium Dioxide | | |
| Hydrophilic | Fumed Silica/ | 0.2 | 5.0 |
| Suspension Aid | Dibasic Calcium Phosphate | | |
| Hydrophilic Diluent | Water/ | 2.0 | 20.0 |
| | Glycerin | | |

Table 3: Key Aspects that Must be Met Alongside the Proposed Ranges Provided in Table 2.

This disclosure is intended to cover formulative percentage ranges outlined above
in Table 2 in conjunction with all the following key aspects listed below.

| Item# | Description |
|---|---|
| 1 | The system is pressurized, absent of propellant in direct contact with the formulation product, and |
| | dispenses liquid in a sprayed or atomized manner. |
| 2 | The system's outer diluent is hydrophobic. (The pH of the system cannot be taken directly.) |
| 3 | The system utilizes at least 1 ingredient from each of the classifications provided at a formulative % |
| | weight which falls within the weight ranges provided. |
| 4 | The system contains at minimum one active ingredient that may be described as a mineral. |
| 5 | The system must be utilized topically for a beneficial purpose. |
| 6 | The table provides N = 2 specific ingredients for each ingredient classification. These are intended to serve |
| | as specific examples. The disclosed system is intended to extend to ingredients not specifically mentioned |
| | but meet the ingredient classifications provided. |
| 7 | The table provided above is intended to serve as the base of the technology. The disclosed formulation is |
| | intended to extend to formulations which utilize additional ingredients and/or ingredient classifications not |
| | mentioned above. |

### Example 3. Testing Various Criteria

Importance of saltwater concentration tested (6.3% failed, 12.6% passed, 19.1% failed).

Increased viscosity is not correlated with increased stability.

Triethoxycaprylylsilane coated zinc oxide remained stable for over 1 Week at 50° C.

Coco-Caprylate resulted in sprayable products.

A stable sprayable formulation can be achieved at both 20% and 25% zinc oxide.

The use of stearalkonium hectorite gel ensures proper dispersal. Bentone Gel IPM V, Bentone Gel GTCC V, Bentone Gel TN V, etc.

Formulas stable at 50° C. for 2+ weeks: Formula #01, Formula #09, Formula #11, Formula #16.

Stable Formulas Passing Spray-Ability Testing: Formula #01, #16, #23, #24.

Formula #01: 20% Zinc Oxide.

Formula #16: 25% Zinc Oxide.

Formula #23: 20% Zinc Oxide (produced undesirable large droplets).

Formula #04: 20% Zinc Oxide.

In-Vitro SPF 30+ results were achieved using 20% w and 25% w zinc oxide formulations.

TABLE 4

| | Mean | Mean | Mean |
|---|---|---|---|
| | SPF | UVAI/ | Critical |
| Formulation | Value | UV Ratio | Wavelength |
| ID250201-23 Lot#060925-238/29 | 34.3 | 17.7 | 373 |
| (20% Zinc Oxide) | | | |
| ID250201-16(B) Lot#061025-238/28 | 45.4 | 21.4 | 373 |
| (25% Zinc Oxide) | | | |
| ID250201-01(E) Lot#061025-238/27 | 35.1 | 17.8 | 373 |
| (20% Zinc Oxide) | | | |

Table 4 In-Vitro SPF results

The formulation listed in Table 5 provides the materials and concentrations of an embodiment of the disclosed formulation. The formula is utilized to create a stable zinc oxide suspension that does not require agitation to resuspend. This ability is paired with spray-ability.

The formulation in Table 5 is known to produce a suspension of 20% zinc oxide that does not sag for T=2 months at 50° C., and produces a fine mist when dispensed from a BOV system.

The Table 5 formulation (working formula) and Table 6 (working formula) processing are utilized for comparison purposes throughout.

TABLE 5
A Working Formula (another embodiment of the disclosed formulation)

| | | | FORMULATION |
|---|---|---|---|
| Phase | TRADE NAME | INCI | WT % |
| A | Capric Caprylic | Capric Caprylic Triglyceride | 20.00 |
| | Triglyceride | | |
| A | Cetiol C5 | Coco-Caprylate | 37.63 |
| A | Dispersun DSP - OL1000 | Polyhydroxy stearic Acid | 2.00 |
| A | Rheopearl TT2 | Dextrin Palmitate/Ethyl hexanoate | 0.50 |
| B | Bentone 27 VCG | Steralkonium Hectorite | 2.80 |
| B | Lexfilm Sun Natural | Capryloyl Glycerin/Sebacic Acid | 1.00 |
| | | Copolymer | |
| B | Assure+ | Pongamia extract | 0.15 |
| C | Super Zinc Shear 1000 | Zinc Oxide + Triethoxycaprylylsilane + | 21.27 |
| | | Ethyl Ferulate | |
| D | Aerosil 200 | Hydrophilic Fumed Silica | 1.25 |
| D | Water | Water | 12.20 |
| D | Salt | Sodium Chloride | 0.40 |
| D | Parfum | Parfum | 0.80 |

TABLE 6
Working Formula Processing Steps

| STEP | Processing Steps Utilized for a 200 g Batch |
|---|---|
| #01 | MAIN: Add items in phase A under a vigorous vortex using an impeller mixer. Allow not |
| | less than (NLT) 2 minutes of mixing between each addition. (Heat to 50° C.) |
| #02 | MAIN: Homogenize the phase A suspension using a small sized stator for 2 minutes at 7000 |
| | rpm (Fine Mesh Stator) |
| #03 | MAIN: Return main phase to vigorous prop mixing. Add phase B ingredients. Allow NLT 2 |
| | minutes of mixing between each addition. |
| #04 | MAIN: Return to homogenizing using a small sized stator for 2 minutes at 7000 rpm (Fine |
| | Mesh Stator) |
| #05 | MAIN: Return main phase to vigorous prop mixing. Add phase C, Allow NLT 2 minutes of |
| | mixing between before continuing. |
| #06 | MAIN: Return to homogenizing using a small sized stator for 2 minutes at 7000 rpm (Fine |
| | Mesh Stator) |
| #07 | MAIN: Return main phase to vigorous prop mixing. Add phase D, Allow NLT 2 minutes of |
| | mixing between before continuing. |
| #08 | MAIN: Return to homogenizing using a small sized stator for 2 minutes at 7000 rpm (Fine |
| | Mesh Stator) |
| #09 | MAIN: Using an anchor mixer under low shear (100 rpm), mix until ambient temperature is |
| | reached (NLT 30 minutes). |
| #10 | Fill bulk into APTAR BOV's at 35-40 psi cut bag pressure. Choose an Actuator appropriately |
| | and grade the resulting spray. |

TABLE 7
Working Formula Aerosol Component Specifications
ORIGINAL WORKING FORMULA AEROSOL
COMPONENT SPECIFICATIONS

| Component/ | |
|---|---|
| Attribute | Specification |
| Crimp | Height: 0.205″ Diameter: 1.07″ CBP: 35-40 psi |
| Specifications | |
| Can | Machine Curl 45 mm × 115 mm (BFC = 160 Ml) |
| BOV | Aptar A3 (35 mm × 88 mm) Max Fill = 77 mL |
| Actuator | Aptar Markus DU4027 non-mechanical breakup 32.5 |

### Formulation Physical Testing Evaluations:

Formulations were monitored by viscosity, stability in stressed conditions, and ability to deliver an atomized spray upon actuation. Only stability in stressed conditions was studied across every formula monitored. Viscosity was only studied on a smaller subset of formulas and spray pattern was only analyzed if the formulations passed a minimum of 1-week in stressed conditions stability.

Viscosity was measured using 2 separate methods. Both methods utilize a Brookfield LV Viscometer with material held at 25° C. contained in a 4 oz jar, an S-62 spindle with readings taken post 1-minute of spinning. The methods differed by spindle rpm which was either 3 rpm or 6 rpm.

Stressed stability utilized 20 mL scintillation vials containing ˜18 mL of material placed upright in a 50° C. chamber and left undisturbed. Vials were visually analyzed daily for the presence or absence of a supernatant. The presence of any visual supernatant constituted stability failure.

Spray pattern analysis was determined by actuating a can for approximately 1-second, approximately 6-inches from a sheet of standard blue printer paper. Both the paper and the can were upright upon actuation. A spray pattern of finely dispersed droplets was considered passing. A spray pattern of larger droplets which create a downward bleeding pattern upon paper contact was considered marginal. A spray pattern which resulted in a dripping pattern upon paper contact or product streaming was considered failure.

### Water Content Evaluation:

Listed below are the results of N=3 separate batches. All N=3 batches utilized the materials listed in Table 5. The concentrations shown in Table 5 were set as the center point. Batches were created in a manner to minimize variability beyond water content. Table 8 provides all % weight changes that occurred between each of the N=3 batches. Results indicate the formula can handle more water than the currently targeted center point.

TABLE 8
Water Content Evaluation

| | CHANGES TO | | VISCOSITY RESULTS | | | | | |
|---|---|---|---|---|---|---|---|---|
| FORMULA/ | FORMULA | | LV, S-62, | | LV, S-62, | | 50° C. | |
| WATER | TRADE | % | 3 rpm, 25° C., | | 6 rpm, 25° C., | | STABILITY | SPRAY- |
| CONTENT | NAME | WEIGHT | 1 min | | 1 min | | RESULT | ABILITY |
| −50% saltwater | Capric | 22.21 | 1490 | cp | 864.8 | cp | FAIL | NA |
| ID250201-(07) | Caprylic | | | | | | (Failed within | |
| | Triglyceride | | | | | | 3 Days) | |
| | Cetiol C5 | 41.72 | | | | | | |
| | Water | 6.10 | | | | | | |
| | Salt | 0.20 | | | | | | |
| ORIGINAL | Capric | 20.00 | 585 | cp | 830 | cp | PASS | PASS |
| FORMULA | Caprylic | | | | | | (1-Month+) | |
| ID250201-(01) | Triglyceride | | | | | | | |
| | Cetiol C5 | 37.63 | | | | | | |
| | Water | 12.20 | | | | | | |
| | Salt | 0.40 | | | | | | |
| +50% Saltwater | Capric | 17.81 | 4799 | cp | 2899 | cp | PASS | MARGINAL |
| ID250201- (08) | Caprylic | | | | | | (Failed at 2 | |
| | Triglyceride | | | | | | Weeks) | |
| | Cetiol C5 | 33.52 | | | | | | |
| | Water | 18.30 | | | | | | |
| | Salt | 0.80 | | | | | | |

### Shear Energy Evaluation:

Listed below are the results of N=3 separate batches. All N=3 batches utilized the materials and concentrations listed in Table 5. Batches were created in a manner such that homogenization time provided was the only changing variable. Results indicate the formula is shear sensitive.

TABLE 9
Shear Energy Evaluation

| | HOMOGENIZATION | | VISCOSITY RESULTS | | | | | |
|---|---|---|---|---|---|---|---|---|
| | TIME | | LV, S-62, | | LV, S-62, | | 50° C. | |
| | Prior to | Post Zinc | 3 rpm, 25° C., | | 6 rpm, 25° C., | | STABILITY | SPRAY- |
| FORMULATION | Zinc Add. | Add. | 1 min | | 1 min | | RESULT | ABILITY |
| ID250201- (01) - | 04 min/ | 04 min/ | 585 | cp | 830 | cp | PASS | PASS |
| 20% ZnO | 7000 rpm | 7000 rpm | | | | | (Min 1- | |
| (Medium Screen | | | | | | | Month) | |
| Stator) | | | | | | | | |
| ID250201- (01C) - | 04 min/ | 04 min/ | 1470 | cp | 950 | cp | PASS | PASS |
| 20% ZnO | 7000 rpm | 7000 rpm | | | | | (Failed at 2 | |
| (Small Screen | | | | | | | Weeks) | |
| Stator) | | | | | | | | |
| ID250201- (01D) - | 04 min/ | 07 min/ | 4139 | cp | 2439 | cp | PASS | PASS |
| 20% ZnO | 7000 rpm | 7000 rpm | | | | | (Failed at 2 | |
| (Small Screen | | | | | | | Weeks) | |
| Stator) | | | | | | | | |
| ID250201- (01B) - | 04 min/ | 10 min/ | 6489 | cp | 4259 | cp | PASS | MARGINAL |
| 20% ZnO | 7000 rpm | 7000 rpm | | | | | (Failed at 2 | |
| (Small Screen | | | | | | | Weeks) | |
| Stator) | | | | | | | | |
| ID250201- (16) - | 04 min/ | 04 min/ | 470 | cp | 709 | cp | PASS | PASS |
| 25% ZnO | 7000 rpm | 7000 rpm | | | | | (2-Weeks+) | |
| (Small Screen | | | | | | | | |
| Stator) | | | | | | | | |

### Zinc Oxide Coating Variations Evaluation:

Listed below are various zinc oxide products that were attempted using the formulation of Table 5. With the exception of zinc oxide source, all other variables were kept constant including batch size, processing times, materials. Results in these experiments indicate that only triethoxycaprylylsilane coated zinc oxide may be utilized to create a stable suspension.

TABLE 10
Zinc Oxide Coatings Evaluation

| | | | | 50° C. | |
|---|---|---|---|---|---|
| | | | ZINC OXIDE COATING | STABILITY | |
| FORMULATION | SUPPLIER | PRODUCT | TESTED | RESULT | SPRAYABILITY |
| ID250201- (01) - | Visor | Super Zinc | Triethoxycaprylylsilane + | PASS | PASS |
| 20% ZnO | | Sheer 1000 | Ethyl Ferulate | (1-Month+) | |
| ID250201- (02B) - | Evercare | Zano 10 Plus | Triethoxycaprylylsilane | PASS | PASS |
| 20% ZnO | | | | (Failed at 2 | |
| | | | | Weeks) | |
| ID250201- (05) - | Sunjin | SunZnO- | Stearic Acid | FAIL | NA |
| 20% ZnO | | 200SA | | | |
| ID250201- (03) - | Kobo | ZNO-C- | Stearoyl Glutamic Acid + | FAIL | NA |
| 20% ZnO | | ASGP4 | Polyhydroxystearic | | |
| | | | Acid | | |
| ID250201- (06) - | Kobo | ZNO-C- | Jojoba Esters | FAIL | NA |
| 20% ZnO | | NJE3 | | | |
| ID250201- (04) - | Zochem | Zoco 102 | Uncoated | FAIL | NA |
| 20% ZnO | | | | | |

### Emollient Ester Variations Evaluation:

Listed below are various zinc oxide products that were attempted using the formulation of Table 5. With the exception of zinc oxide source, all other variables were kept constant including batch size, processing times, materials. Results of these experiments indicate that only Triethoxycaprylylsilane coated zinc oxide may be utilized to create a stable suspension.

TABLE 11
Emollient Ester Variations Evaluation

| | CHANGES TO FORMULA | | 50° C. | |
|---|---|---|---|---|
| | | FORMULATION | STABILITY | SPRAY- |
| FORMULATION | INGREDIENTS | WEIGHT % | RESULT | ABILITY |
| ORIGINAL | Capric Caprylic Triglyceride | 20.00 | PASS | PASS |
| FORMULA | Coco-Caprylate | 37.63 | (1-Month+) | |
| ID250201- (01) - | | | | |
| 20% ZnO | | | | |
| ID250201- (09) - | Capric Caprylic Triglyceride | 33.52 | PASS | FAIL, |
| 20% ZnO | Coco-Caprylate | 17.81 | | Streaming, No |
| | | | | Misting |
| ID250201- (10) - | Capric Caprylic Triglyceride | 20.00 | FAIL | NA |
| 20% ZnO | Hemi-Squalane (C₁₃-C₁₅ Alkanes) | 37.63 | | |
| ID250201- (11) - | Capric Caprylic Triglyceride | 17.81 | PASS | FAIL, |
| 20% ZnO | Natpure Feel CCC (Coco- | 33.52 | | Streaming, No |
| | Caprylate/Caprate) | | | Misting |
| ID250201- (12) - | Capric Caprylic Triglyceride | 17.81 | PASS | FAIL, |
| 20% ZnO | Floraesters IPJ (Isopropyl | 33.52 | (Failed at 2 | Too thick to |
| | Jojobate + Jojoba Alcohol + | | Weeks) | Spray |
| | Jojoba Esters) | | | |
| ID250201- (17) - | Capric Caprylic Triglyceride | 17.81 | PASS | FAIL, |
| 20% ZnO | Hallsens DIAS (Diisoamyl | 33.52 | (Failed at 2 | Too thick to |
| | Succinate) | | Weeks) | Spray |

### Zinc Oxide Concentration Evaluation:

Listed below are formulations which contain zinc oxide at either 20% w/w or 25% w/w. These concentrations are important because zinc oxide rarely provides SPF30+ protection at levels below 20% w/w and 25% w/w is the maximum zinc oxide content allowed within an OTC SPF product. Additionally, 25% zinc oxide is a common concentration utilized for OTC barrier protection. (Note, 40% w/w Zinc Oxide is the FDA OTC limit for barrier protection).

TABLE 12
Zinc Oxide Concentration Evaluation

| | | CHANGES TO FORMULA | | Zinc | 50° C. | |
|---|---|---|---|---|---|---|
| | ZINC | | FORMULATION | Oxide | STABILITY | SPRAY- |
| FORMULATION | PRODUCT | INGREDIENT | WEIGHT % | Content | RESULT | ABILITY |
| ORIGINAL | Super | Capric Caprylic | 20.00 | 20% | PASS | PASS |
| FORMULA | Zinc | Triglyceride | | | (1-Month+) | |
| ID250201- | Sheer | Cetiol C5 | 37.63 | | | |
| (01) | 1000 | Rheopearl TT2 | 0.50 | | | |
| | | Aerosil 200 | 1.25 | | | |
| | | Water | 12.20 | | | |
| | | Sodium Chloride | 0.40 | | | |
| ID250201- | Super | Capric Caprylic | 15.96 | 25% | PASS | FAIL |
| (13) | Zinc | Triglyceride | | | (Failed at 2 | Too thick to |
| | Sheer | Cetiol C5 | 30.04 | | Weeks) | Spray |
| | 1000 | Rheopearl TT2 | 0.50 | | | |
| | | Aerosil 200 | 1.25 | | | |
| | | Water | 18.30 | | | |
| | | Sodium | 0.60 | | | |
| | | Chloride | | | | |

TABLE 1
Zinc Oxide Evaluation Continued
Table Continuation:

| | | CHANGES TO FORMULA | | Zinc | 50° C. | |
|---|---|---|---|---|---|---|
| | ZINC | | FORMULATION | Oxide | STABILITY | SPRAY- |
| FORMULATION | PRODUCT | INGREDIENT | WEIGHT % | Content | RESULT | ABILITY |
| ID250201- | Super | Capric Caprylic | 18.15 | 25% | PASS | FAIL |
| (14) | Zinc | Triglyceride | | | (Failed at 2 | Too thick |
| | Sheer | Cetiol C5 | 34.15 | | Weeks) | to Spray |
| | 1000 | Rheopearl TT2 | 0.50 | | | |
| | | Aerosil 200 | 1.25 | | | |
| | | Water | 12.20 | | | |
| | | Sodium Chloride | 0.40 | | | |
| AID250201- | Super | Capric Caprylic | 18.38 | 25% | PASS | FAIL |
| (15) | Zinc | Triglyceride | | | | Too thick |
| | Sheer | Cetiol C5 | 34.57 | | | to Spray |
| | 1000 | Rheopearl TT2 | 0.50 | | | |
| | | Aerosil 200 | 0.60 | | | |
| | | Water | 12.20 | | | |
| | | Sodium Chloride | 0.40 | | | |
| ID250201- | Super | Capric Caprylic | 18.38 | 25% | Pass | PASS |
| (16) | Zinc | Triglyceride | | | (2-Week+) | |
| | Sheer | Cetiol C5 | 35.07 | | | |
| | 1000 | Rheopearl TT2 | 0.00 | | | |
| | | Aerosil 200 | 0.60 | | | |
| | | Water | 12.20 | | | |
| | | Sodium Chloride | 0.40 | | | |

### Stearalkonium Hectorite Dispersal Evaluation:

Listed below are formulations which utilize different methods and additives to disperse stearalkonium hectorite.

TABLE 2
Stearalkonium Hectorite Dispersal Evaluation

| | | 50° C. | |
|---|---|---|---|
| | | STABILITY | SPRAY- |
| FORMULA | STEARALKONIUM HECTORITE DISPERAL SPECIFICS | RESULT | ABILITY |
| ID250201- | CCT, Coco-Caprylate, Polyhydroxy Stearic Acid, | PASS | PASS |
| (01) | Dextrin Palmitate/Ethyl Hexanoate added using low | (1-Month+) | |
| 20% ZnO | shear + 50° C. heat | | |
| | Stearalkonium hectorite added under low shear @ | | |
| | 50° C. | | |
| | Homogenize @ 7000 rpm using medium screen for 2 | | |
| | min. | | |
| | Zinc Oxide added under low shear@ 50° C. | | |
| | Homogenize @ 7000 rpm using medium screen for 2 | | |
| | min. | | |
| | Hydrophilic silica + saltwater added under low shear | | |
| | @ 50° C. | | |
| | Homogenize @ 7000 rpm using medium screen for 2 | | |
| | min. | | |
| ID250201- | CCT, Coco-Caprylate, Polyhydroxy Stearic Acid, | FAIL | N/A |
| (21) | Dextrin Palmitate/Ethyl Hexanoate added using low | | |
| 20% ZnO | shear + 50° C. heat | | |
| | Homogenize @ 7000 rpm using medium screen for 2 | | |
| | min. | | |
| | Hydrophilic silica + saltwater added under low shear | | |
| | @ 50° C. | | |
| | Homogenize @ 7000 rpm using medium screen for 2 | | |
| | min. | | |
| | Stearalkonium hectorite added under low shear @ | | |
| | 50° C. | | |
| | Zinc Oxide added under low shear@ 50° C. | | |
| | Homogenize @ 7000 rpm using medium screen for 2 | | |
| | min. | | |
| ID250201- | Utilize predispersed stearalkonium hectorite available as | FAIL | N/A |
| (22) | Bentone Ultimate LC | | |
| 20% ZnO | | | |
| ID250201- | Utilized 8.0% Bentone Lux XO (C13-C15 Alkanes + | PASS | MARGINAL |
| (23) | Disteardimonium Hectorite + Polygyceryl-3 | (1-Week+) | Large Droplets |
| 20% ZnO | Polyricinonleate) in place of 2.8% Stearalkonium | | |
| | Hectorite and 5.2% CCT | | |
| ID250201- | Utilized 8.0% Bentone GEL GTCC V (CCT + | PASS | PASS |
| (24) | Stearalkonium Hectorite + Propylene Carbonate) in | (1-Week+) | |
| 20% ZnO | place of 2.8% Stearalkonium Hectorite and 5.2% CCT | | |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A formulation comprising a homogeneous suspension of a uniform composition of solid particulates comprising zinc oxide or titanium dioxide, or a combination of both, suspended in an oleophilic system possessing a positive Octanol Water Partition Coefficient, wherein the formulation does not comprise an emulsifier, wherein the formulation is expelled from a dispensing container system without void space, wherein said suspension does not comprise particulate settling of the homogeneous suspension of a uniform composition, wherein more than 70% of the solid particulates remains suspended through at least six months undisturbed.

2. (canceled)

3. The formulation of claim 1, wherein the homogeneous suspension further comprises one or more solid particulates selected from the group consisting of stearalkonium hectorite gel, bentonite, hectorite, iron oxide, mica, hydroxyapatite, kaolin, clay, stearalkonium hectorite, and distarch phosphate.

4. The formulation of claim 3, wherein the formulation comprises stearalkonium hectorite gel.

5. The formulation of claim 1, wherein coco-caprylate, and/or C₁₂-C₁₅ Alkyl Benzoates are present within the outer phase of the formulation and utilized above 20% of the formulation's weight.

6. The formulation of claim 1, comprising coco-caprylate.

7. The formulation of claim 1, wherein water containing chloride salts, or sulfate salts are present within the formulation inner phase below 20% formulation's weight.

8. The formulation of claim 1, wherein the zinc oxide is present from 0% to 40% of the formulation's weight and/or wherein the titanium dioxide is present from 0% to 25% of the formulation's weight.

9. (canceled)

10. The formulation of claim 1, comprising SPF 30+ and zinc oxide from 20% to 25% of the formulation weight.

11-12. (canceled)

13. The formulation of claim 1, comprising 12.6% saltwater.

14. The formulation of claim 1, comprising triethoxycaprylylsilane coated zinc oxide and/or titanium dioxide.

15. The formulation of claim 1, comprising the following:

| | Specific Ingredients | Lower | Upper |
|---|---|---|---|
| | (N = 2 Provided per | % Weight | % Weight |
| Ingredient Class | Class) | Limit | Limit |
| Emollient Ester | C₁₂₋₁₅ Alkyl Benzoate/ | 20.0 | 90.0 |
| | Coco-Caprylate | | |
| Mineral Dispersant | Polyhydroxystearic Acid/ | 0.5 | 10.0 |
| | Polyamide-8 | | |
| Oil Suspension Aid | Steralkonium Hectorite/ | 0.2 | 10.0 |
| | Ethyl Cellulose | | |
| Mineral Active | Zinc Oxide/ | 5.0 | 40.0 |
| | Titanium Dioxide | | |
| Hydrophilic | Fumed Silica/ | 0.2 | 5.0 |
| Suspension Aid | Dibasic Calcium Phosphate | | |
| Hydrophilic Diluent | Water/ | 2.0 | 20.0. |
| | Glycerin | | |

16. The formulation of claim 1, wherein said suspension does not comprise particulate settling of the zinc oxide or titanium dioxide, or a combination of both, above about 10% formulation weight.

17. The formulation of claim 1, wherein said formulation's average aerosolized droplet size is less than 50 microns using a volume distribution measured by laser diffraction or microscopy.

18. The formulation of claim 1, wherein the dispensing container system does not need to be shaken before the formulation is expelled from the dispensing container system.

19. The formulation of claim 1, wherein said formulation is for topical, cosmetic, drug, or medical device application.

20-23. (canceled)

24. A dispensing container system comprising a formulation of claim 1.

25. The dispensing container system of claim 24, wherein the container dispensing system without void space is a bag on valve (BOV) pressurized assembly package, comprising a two-way fill/dispensing valve, an attached internal high barrier bag affixed to said valve, and rigid container adapted to holding positive pressure affixed to the valve.

26. The dispensing container system of claim 25, wherein said container is glass, barrier resin, metal/alloy, or another material capable of holding positive pressure.

27. The dispensing container system of claim 25, wherein the BOV pressurized assembly expels the formulation in a metered dispensing system.

28. The dispensing container system of claim 24, wherein the container dispensing system without void space is an Airopack system, a piston can system, a compression sleeve BOV system, or a rubber tube over bag system.

29. A method of preparing a formulation, comprising:
Adding oils and emollient esters (Capric Caprylic Triglyceride/C₁₂₋₁₅ Alkyl
Benzoate/Coco-Caprylate) to a vessel;
applying a vigorous vortex using an impeller mixer;
allowing no less than two minutes of mixing between each addition;
Adding Mineral dispersants to the vessel;
Heating the vessel to 50° C.;
Homogenizing the vessel using a medium sized stator for two minutes at 7000
rpm;
Stopping homogenizing and returning vessel to vigorous prop mixing;
Adding suspending solids and rheology modifiers to the vessel;
allowing no less than two minutes of mixing between each addition;
Returning vessel to homogenizing using a medium sized stator for two minutes at
7000 rpm;
Stopping homogenizing and returning vessel to vigorous prop mixing;
Adding active ingredients (Zinc Oxide/Titanium Dioxide);
Allowing no less than two minutes of mixing between each addition;
Returning vessel to homogenizing using a medium sized stator for five minutes at
7000 rpm;
Stopping homogenizing and returning vessel to vigorous prop mixing;
Adding hydrophilic ingredients including fumed silica and ionized (salted) water
to the vessel; Allowing no less than two minutes of mixing between each addition;
Returning to homogenizing using a medium sized stator for five minutes at 7000
rpm;
Stopping homogenizing;
Using an anchor mixer under low shear, mixing until batch cools from 50° C. to
room temperature; thereby completing bulk processing and resulting in bulk;
Filling bulk into BOV systems at 35-40 psi cut bag pressure.
